SEQUENCE LISTING (6 sequences)

SEQ ID NO: 1 - DNA, 572 bp, Medicago sativa
acagacgaacaataattccaaacacaaccctttaacctttaacttcgaaattcaatttttattctctctgcaacttttcacttttctttctccttttaagatccaacaacgttagcgccgttgtttttttcacaagcaatttcttctgtttaatcttatttttcaattattgttgctttcattccaagttttccttggaaattgaaacttacgttactttaagatttctccaagcttctttttgatgatttttcttggttttactttgattagagggttatggactgttcttctttgtgattaggttatggtgctttatgattattgattgattatcatggtttttttttggggattttgtctaggttatatttgggattgaaaaaaacttttttttttttgctttgtttttaatttgtggtttttctttcttccagaatatatataaggtgatttggggtttgttttattttgaaaaattggggattgattaggttctgggttttgaatttaagttttgagggtgagatttgtcaattgggaattgattaggttttgggttttttttgagt

SEQ ID NO: 2 - DNA, 2344 bp, Medicago sativa
aaaaagtttttcatttattatatttattttttatgtgattatttattatatatataatttagaaaatagaaaaattagagagactgaaatattttatttattaaataaactgatgctttgtttcctggtcaaaaaaaataatttgatgcctttattctagtaagtaattattttttcctaaaaatcaacaataccgttgacccttctgctcaggaaaaatattatgcagaagagaaaaagcctggctagaaacaaagaaagaattatcaacgcgtttcacagacgaacaataattccaaacacaaccctttaacctttaacttcgaaattcaatttttattctctctgcaacttttcacttttctttctccttttaagatccaacaacgttagcgccgttgtttttttcacaagcaatttcttctgtttaatcttatttttcaattattgttgctttcattccaagttttccttggaaattgaaacttacgttactttaagatttctccaagcttctttttgatgatttttcttggttttactttgattagagggttatggactgttcttctttgtgattaggttatggtgctttatgattattgattgattatcatggtttttttttggggattttgtctaggttatatttgggattgaaaaaaacttttttttttttgctttgtttttaatttgtggtttttctttcttccagaatatatataaggtgatttggggtttgttttattttgaaaaattggggattgattaggttctgggttttgaatttaagttttgagggtgagatttgtcaattgggaattgattaggttttgggttttttttgagtatggatgaagctggaagttcaagttctctgcctccttttctggcaaagacttatgaaatggtggatgatcgatcctcggatcctattgtttcgtggagtgcatcgaacaaaagtttcgttgtgtggaatcctccggaattcgcaagggttttgctttccgagattcttaagcacaataacttctccagttttatcaggcagctgaatacatatggatttaggaaagttgatgtcggtgttgtatttgttgatgcgaattcttgttttatgtttattttatttactgattctggtttttggttgcttgaactgattcatagtatatgtatgtgcagccggagcagtgggaatttgccaatgatgattttataagggggcaaccgcatctgatgaagaatattcatagacggaagccggttcatagtcattctctgcataatcttcaagcacaagcctctctaacggagtctgaaaggcagagtatgattgatgaaatagagaagctgaaacaggatagagaacaactcctcgtggagacgaagagataccagcatgattgggaaaggcatgagattcaaatgcattgttcgaaagatcagttggaaaaattggaacataaacaacaaaagatgttgtcctctgtttctgaagcattgcagaaacctatgattgctgtaaatctcttgccactggcagaagccatggagagaaaacgaaggttgccggcgagaagtgtttgctttaataatgaagctagtgttgaagatgctatggagacttctgtagctttgcccagggaaaattcagaggataacttctactttgacgttaaacaccgagagattggatcaacttcgaggcatccgtggcattttgggaagactcttgcacatgaagttggcgacaatttcgttcacactcattcaaacatggatttagatgaatctacatgctgtgctgataggttgtcgatatcttgtcagcaactagacggtgaagttcgacctaagtcacctgaaattgacatgaacgttgaaccagctgctgctgctcttgaggctgttgcagtaaaagaacaacctgcgagaatcaccactgcagcaactggggttaatgatgtattctgggaacagttcttgacagaggatcctggtgcatcggaagcacaggaagtgcaatcagaaagaaaagataatagtagcagaaaaaatgaaggaaaacctagtgatcatggtagattttggtggaacatgcggaaatcaaacaatcatccagaacagatggggcatgttagtcaagtagagaaaatttagtagctattgattatttcttctggttgtgttgccccacaatcatgtgtaataaaacttggtatgcattgtttttgtctgcaatagatacatatttcatactttggctgaggtgaatgttatcaaagattgtgtcactccattggtttttgtctgcaatagatacatattgtttttgtctgca

SEQ ID NO: 3 - DNA, 1979 bp, Artificial
  cDNA sequence
acagacgaacaataattccaaacacaaccctttaacctttaacttcgaaattcaatttttattctctctgcaacttttcacttttctttctccttttaagatccaacaacgttagcgccgttgtttttttcacaagcaatttcttctgtttaatcttatttttcaattattgttgctttcattccaagttttccttggaaattgaaacttacgttactttaagatttctccaagcttctttttgatgatttttcttggttttactttgattagagggttatggactgttcttctttgtgattaggttatggtgctttatgattattgattgattatcatggtttttttttggggattttgtctaggttatatttgggattgaaaaaaacttttttttttttgctttgtttttaatttgtggtttttctttcttccagaatatatataaggtgatttggggtttgttttattttgaaaaattggggattgattaggttctgggttttgaatttaagttttgagggtgagatttgtcaattgggaattgattaggttttgggttttttttgagtatggatgaagctggaagttcaagttctctgcctccttttctggcaaagacttatgaaatggtggatgatcgatcctcggatcctattgtttcgtggagtgcatcgaacaaaagtttcgttgtgtggaatcctccggaattcgcaagggttttgctttccgagattcttaagcacaataacttctccagttttatcaggcagctgaatacatatggatttaggaaagttgatccggagcagtgggaatttgccaatgatgattttataagggggcaaccgcatctgatgaagaatattcatagacggaagccggttcatagtcattctctgcataatcttcaagcacaagcctctctaacggagtctgaaaggcagagtatgattgatgaaatagagaagctgaaacaggatagagaacaactcctcgtggagacgaagagataccagcatgattgggaaaggcatgagattcaaatgcattgttcgaaagatcagttggaaaaattggaacataaacaacaaaagatgttgtcctctgtttctgaagcattgcagaaacctatgattgctgtaaatctcttgccactggcagaagccatggagagaaaacgaaggttgccggcgagaagtgtttgctttaataatgaagctagtgttgaagatgctatggagacttctgtagctttgcccagggaaaattcagaggataacttctactttgacgttaaacaccgagagattggatcaacttcgaggcatccgtggcattttgggaagactcttgcacatgaagttggcgacaatttcgttcacactcattcaaacatggatttagatgaatctacatgctgtgctgataggttgtcgatatcttgtcagcaactagacggtgaagttcgacctaagtcacctgaaattgacatgaacgttgaaccagctgctgctgctcttgaggctgttgcagtaaaagaacaacctgcgagaatcaccactgcagcaactggggttaatgatgtattctgggaacagttcttgacagaggatcctggtgcatcggaagcacaggaagtgcaatcagaaagaaaagataatagtagcagaaaaaatgaaggaaaacctagtgatcatggtagattttggtggaacatgcggaaatcaaacaatcatccagaacagatggggcatgttagtcaagtagagaaaatttagtagctattgattatttcttctggttgtgttgccccacaatcatgtgtaataaaacttggtatgcattgtttttgtctgcaatagatacatatttcatactttggctgaggtgaatgttatcaaagattgtgtcactccattggtttttgtctgcaatagatacatattgtttttgtctgcaaaaaaaaaaaaaaaaaa

SEQ ID NO: 4 - protein, 402 aa, Medicago sativa
MDEAGSSSSLPPFLAKTYEMVDDRSSDPIVSWSASNKSFVVWNPPEFARVLLSEILKHNNFSSFIRQLNTYGFRKVDPEQWEFANDDFIRGQPHLMKNIHRRKPVHSHSLHNLQAQASLTESERQSMIDEIEKLKQDREQLLVETKRYQHDWERHEIQMHCSKDQLEKLEHKQQKMLSSVSEALQKPMIAVNLLPLAEAMERKRRLPARSVCFNNEASVEDAMETSVALPRENSEDNFYFDVKHREIGSTSRHPWHFGKTLAHEVGDNFVHTHSNMDLDESTCCADRLSISCQQLDGEVRPKSPEIDMNVEPAAAALEAVAVKEQPARITTAATGVNDVFWEQFLTEDPGASEAQEVQSERKDNSSRKNEGKPSDHGRFWWNMRKSNNHPEQMGHVSQVEKI

SEQ ID NO: 5 - protein, 11 aa, artificial
  T7 epitope tag
MASMTGGQQMG

SEQ ID NO: 6 - DNA, 112 bp, Medicago sativa
tttgttttattttgaaaaattggggattgattaggttctgggttttgaatttaagttttgagggtgagatttgtcaattgggaattgattaggttttgggttttttttgagt

(19) United States
(12) Patent Application Publication
(10) Pub. No.: US 2011/0131680 A1
(43) Pub. Date: Jun. 2, 2011

(54) METHOD OF ENHANCING GENE EXPRESSION IN PLANTS

(76) Inventors: Stephen Bowley, Guelph (CA); Helen Byczko, Guelph (CA); Jeremy Friedberg, Toronto (CA)

(21) Appl. No.: 12/882,692

(22) Filed: Sep. 15, 2010

Related U.S. Application Data
(60) Provisional application No. 61/272,340, filed on Sep. 15, 2009.

(51) Int. Cl. C12N 15/87; C12N 5/10

(52) U.S. Cl. 800/278; 435/419

(57) ABSTRACT

A method of enhancing the expression of a gene in a legume plant is provided comprising introducing into the cells of said plant a construct comprising at least a portion of a 5′-untranslated region of a heat shock transcription factor gene from Medicago sativa (MsHsfA4) upstream of a gene to be expressed. Plant cells capable of expressing enhanced levels of a gene are also provided. The cell comprises a construct comprising at least a portion of the 5′-UTR of the MsHsfA4 gene upstream of the gene to be expressed.

### FIELD OF THE INVENTION

The present invention relates generally to the upregulation of gene expression in plants, and more particularly, to the use of a 5′ untranslated gene sequence (UTR) to enhance gene expression in plants.

### BACKGROUND OF THE INVENTION

Alfalfa (Medicago sativa L.), also known as Lucerne, is an important legume that is cultivated based on its versatility and its value as a nutrient-rich forage crop. Its attributes include rapid field establishment, high biomass yield, and good disease resistance. For livestock producers, alfalfa is highly valued for animal feed because of its high protein, vitamin content, high intake potential, and digestibility. This crop also has an important role in sustainable production systems due to its soil improvement and nitrogen-fixing symbioses with Rhizobia, a soil bacterium.

Alfalfa is one of the most globally versatile crops and is the third largest crop by area in Canada with over 4.5 million hectares under production. Canada is also the world's leading exporter of alfalfa pellets (350,000 tons annually) and second largest exporter of alfalfa cubes (225,000 tons annually). The export of certified alfalfa seed totaled 5.2 million kilograms in 2001/02. The greatest challenge for alfalfa growers is to maintain profitability and this requires alfalfa stands to continue high yields over several years. Since yield depends on stand density, most alfalfa stands are expected to last between 3-6 years, but all too often by the third production year alfalfa has thinned out substantially due to environmental stresses such as winterkill. Winterkill in alfalfa is classified as the failure of an over wintering crop to survive extremely low temperatures and cold injury caused by repeated freezing and thawing, and subsequent death of a plant. Many cumulative factors such as disease and pest injury and warm periods of 10° C. weather in late winter along with repeated freezing and thawing can result in a break in alfalfa's dormancy and render it susceptible to damage.

Genetic improvement of stress tolerance has traditionally been problematic since stress-related genes are complex and involve a network of biochemical pathways. Fortunately, recent developments in plant tissue culture and gene transformation techniques offer new opportunities over conventional breeding enabling the improvement of forage crops such as alfalfa. Manipulation of stress tolerance systems through biotechnology may provide improvements in abiotic stress tolerance of alfalfa.

Abiotic stress is a major limiting factor in agricultural crop production in many countries. The major abiotic stresses of economic importance include heat shock, cold shock, anoxia, drought/desiccation, high salinity, organic and inorganic pollutants. Cellular responses to these abiotic stresses are evolutionarily conserved from bacteria, plants to mammalian species. These ubiquitous responses are sophisticated mechanisms which aid to minimize and correct cellular damage so that organisms are able to acclimate, survive and reproduce within their changing environment. Sessile organisms like plants rely on highly sensitive signal detection, transduction and adaptation mechanisms to adjust to such environmental fluctuations. The most common environmental stress that will influence growth and sustainability are conditions of heat shock/drought, cold shock and high salinity, all of which have similar physiological consequences of desiccation to plant cells.

A number of scientists have focused their attempts to engineer and enhance overall stress tolerance in plants by improving a plant's ability to tolerate stressors such as heat. The acclimation or tolerance to heat stress (HS) was determined to be correlated with the activation of a heat shock gene response.

Indeed, for many plant species the synthesis of heat shock proteins (Hsps) has been speculated to be correlated with the acquisition of thermotolerance. Increased levels of low molecular weight (lmw) HSPs were found to exist in cultivars of wheat that were heat tolerant as compared to cultivars that were less heat tolerant. In alfalfa, non-dormant varieties are more adapted to warmer and/or drier climates than dormant varieties. From chamber exposure experiments, a pattern that displayed a delay in expression initiation and a decrease in the magnitude of transcript levels was observed in alfalfa cultivars. This pattern was found to relate to dormancy. In this regard, it is believed that, with alfalfa, non-dormant cultivars may be less dependent on the heat shock response for heat protection, and thus require less induction of Hsps compared to dormant cultivars whose physiology is more adapted to colder climates.

Regulation of heat shock proteins in plants has been researched and a correlative relationship has been found between shock transcription factors (Hsf) gene expression and lmwHsp expression. The most conserved part of Hsfs is the DNA binding domain (DBD) located near the N-terminus. The DBD consists of helix-turn-helix motif (H2-T-H3) with a hydrophobic core required for recognition and binding of palindromic heat shock elements (HSEs) (5′-AGAAnnTTCT-3′) in the promoter region of Hsp genes. The DBD of all plant Hsf genes sequenced to date contain an intron, located immediately downstream of the H2-T-H3 motif. Directly C-terminus of the DBD, are two adjacent hydrophobic heptad repeats (HR-A and HR-B) which are separated by a flexible linker peptide of varying length and comprise the oligomerization domain. The role of the HR-A/B may be to provide hydrophobic surfaces for Hsf trimerization.

In plants, Hsfs have been categorized into 3 protein family classes (classes A, B, and C) based on the variation of their flexible linkers, HR-A/B regions and parsimony analysis of their DNA-binding domains (DBDs). The major classes A (activation) and B (attenuation) Hsfs appear to be unique to plants. There is evidence that the tomato HsfBl exists as a dimer, whereas HsfA1 and HsfA2 are trimers. Trimerization of HSF via the formation of a triple stranded alpha-helical coil is a prerequisite for high affinity DNA binding and transcriptional activation of Hsp genes. The nuclear localization signal (NLS) is essential for nuclear uptake of the Hsf protein. A less conserved region of the Hsf gene is the C-terminal activator domain (CTAD) which is rich in aromatic, hydrophobic and acidic amino acids (the AHA motifs). The AHA motifs have been determined essential for transcriptional activation for class A Hsfs; in contrast, however, they are absent in class B Hsfs.

Of course, it is to be understood that the stressor itself will determine which gene is involved in modulating the response to the stressor. As the modulation of heat shock protein expression is correlated with the acquisition of thermotolerance, the modulation of the expression of enzymes associated with the Halliwell-Asada pathway (e.g. ascorbate peroxidase, glutathione reductase, superoxide dismutase, etc.) is correlated with oxidative stress tolerance.

Given the foregoing, it would be desirable to manipulate the expression of genes correlated to a stress-response in plants, in order to improve common abiotic stress tolerance of agronomically important plant systems.

### SUMMARY OF INVENTION

It has now been determined that the 5′ non-coding sequence of heat shock transcription factor A4, such as the 5′-UTR in Medicago sativa (MsHsfA4), may be used to enhance the transcription of a downstream gene in legume plants.

Thus, in one aspect of the invention, a method of enhancing the expression of a gene to be expressed in a legume plant is provided comprising introducing into the cells of said plant a construct comprising at least a portion of the 5′-UTR of the HsfA4 gene, such as the MsHsfA4 gene, upstream of the gene to be expressed.

A recombinant plant cell capable of exhibiting enhanced expression of a gene, wherein said cell comprises at least a portion of the 5′-UTR of the HsfA4 gene, such as the MsHsfA4 gene, upstream of the gene to be expressed

These and other aspects of the invention will become apparent by reference to the detailed description and figures.

### BRIEF DESCRIPTION OF THE FIGURES

FIG. 1 is a schematic of control effector constructs, Hsf effector constructs, internal standard and reporter vector constructs utilized to assess Gus reporter gene expression;

FIG. 2 is a schematic illustrating alignment of homologous gene segments of MsHsfA4 in comparison to Phaseolus, Nicotiana, and Arabidopsis class A4 Hsfs. ((+1) transcription start site, (ATG) translation start site, (ORF) open reading frame (UTR) untranslated region);

FIG. 3 graphically illustrates PEG-mediated transformation of Arabidopsis thaliana and Medicago sativa Hsfs co-expressed with a 9xHSE/GUS reporter in tobacco protoplast system expressed as mean GUS activity as a percentage of the endogenous level (At1 (AtHsfA1), At4 (AtHsfA4), Ms4 (MsHsfA4a), U-Ms4 (MsHsfA4a+5′-UTR));

FIG. 4 graphically illustrates PEG-mediated transformation of Arabidopsis thaliana and Medicago sativa Hsfs co-expressed with a 9×HSE/GUS reporter in tobacco, Arabidopsis, Alfalfa, Medicago truncatula, Lotus japonicus protoplast systems, expressed as a mean percentage of MsHsfA4;

FIG. 5 graphically illustrates PEG-mediated transformation of Arabidopsis thaliana and Medicago sativa Hsfs co-expressed with a 9×HSE/GUS reporter in Arabidopsis protoplast system expressed as mean GUS activity as a percentage of the endogenous level;

FIG. 6 graphically illustrates PEG-mediated transformation of Arabidopsis thaliana and Medicago sativa Hsfs co-expressed with a 9×HSE/GUS reporter in Medicago sativa (alfalfa) protoplast system expressed as mean GUS activity as a percentage of the endogenous level;

FIG. 7 graphically illustrates PEG-mediated transformation of Arabidopsis thaliana and Medicago sativa Hsfs co-expressed with a 9×HSE/GUS reporter in Medicago truncatula protoplast system expressed as mean GUS activity as a percentage of the endogenous level;

FIG. 8 graphically illustrates PEG-mediated transformation of Arabidopsis thaliana and Medicago sativa Hsfs co-expressed with a 9×HSE/GUS reporter in Lotus japonicus (Trefoil) protoplast system expressed as mean GUS activity as a percentage of the endogenous level;

FIG. 9 illustrates the genomic sequence of the 5 ′-UTR of MsHSFA4, corresponding to SEQ ID NO:1;

FIG. 10 illustrates MsHSFA4 genomic sequence (GenBank Accession Number AF494082) corresponding to SEQ ID NO:2; and

FIG. 11 illustrates the cDNA sequence of MsHSFA4 (GenBank Accession Number AF235958) including the 5′-UTR, corresponding to SEQ ID NO:3.

### DETAILED DESCRIPTION OF THE INVENTION

A method of enhancing the expression of a gene in a legume plant is provided comprising introducing into the cells of said plant a nucleotide construct comprising at least a portion of the 5′-UTR of the HsfA4 gene, e.g. MsHsfA4, upstream of the gene to be expressed.

The terms “enhancing” and “upregulating” are used interchangeably herein and refer to expression of a gene that exceeds the endogenous level of expression, i.e. an expression level achieved in the absence of the presence of an upstream HsfA 5′-UTR, e.g. the 5′-UTR of MsHsfA4.

The term “legume plant” is used herein to refer to plants such as members of the Fabaceae family such as Medicago ssp. including Medicago sativa (alfalfa) and Medicago truncatula (barrel clover), Lotus ssp including Lotus japonicus, Phaseolus acutifolius (bean) and other Phaseolus sub-species (ssp.), Pisum sativum (pea), Trifolium ssp. (clover) and Glycine max (soy).

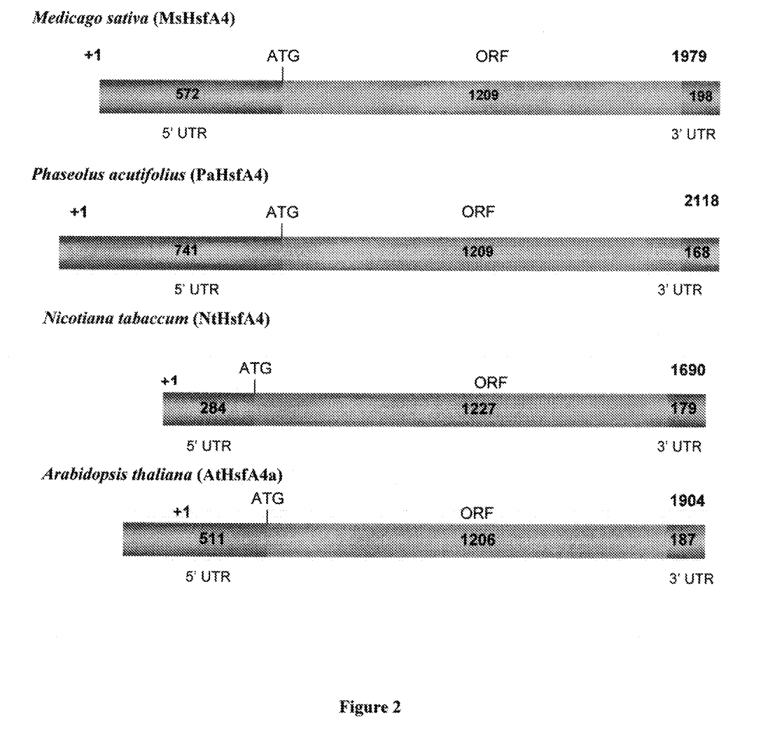

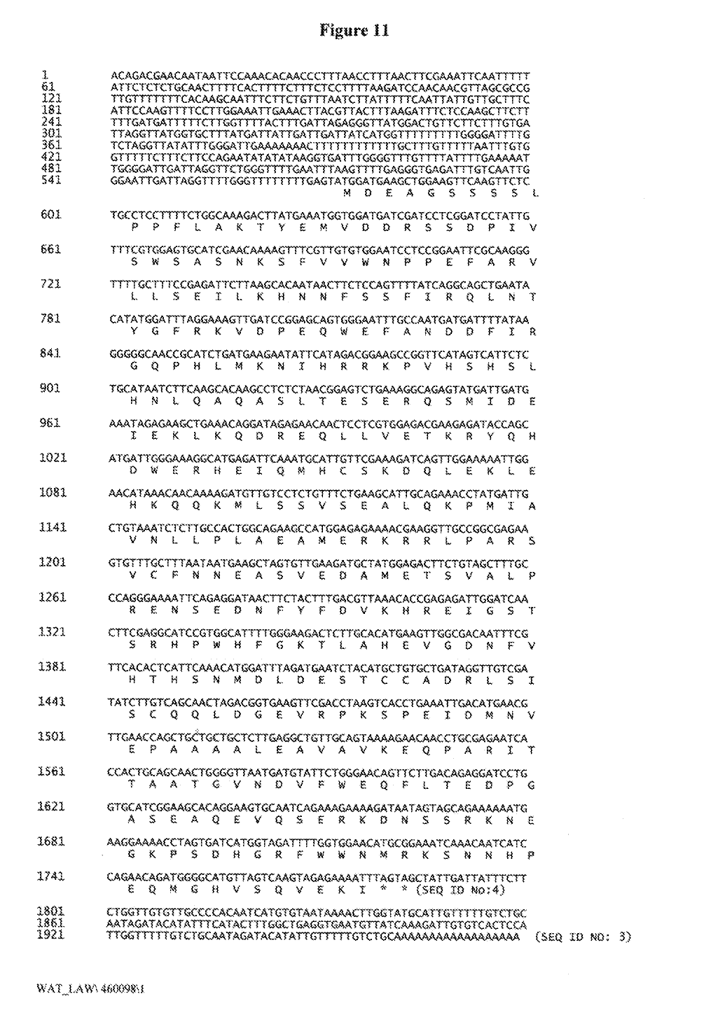

The term “HsfA4” refers to a class of heat shock protein transcription factors (Hsfs) that activate transcription of heat shock proteins in plants, namely Class A heat shock protein transcription factors. Examples of suitable HsfA4 include, HsfA4 such as Medicago sativa HsfA4 (MsHsfA4), the cDNA and genomic DNA sequences of which are provided in FIGS. 10 (SEQ ID NO:2) and 11 (SEQ ID NO:3). Also included, as shown in FIG. 2, are HsfA4 genes from other legume plants such as Phaseolus acutifolius (namely, PaHsfA4), Nicotiana tabaccum (namely, NtHsfA4) and Arabidopsis thaliana (namely, AtHsf A4a).

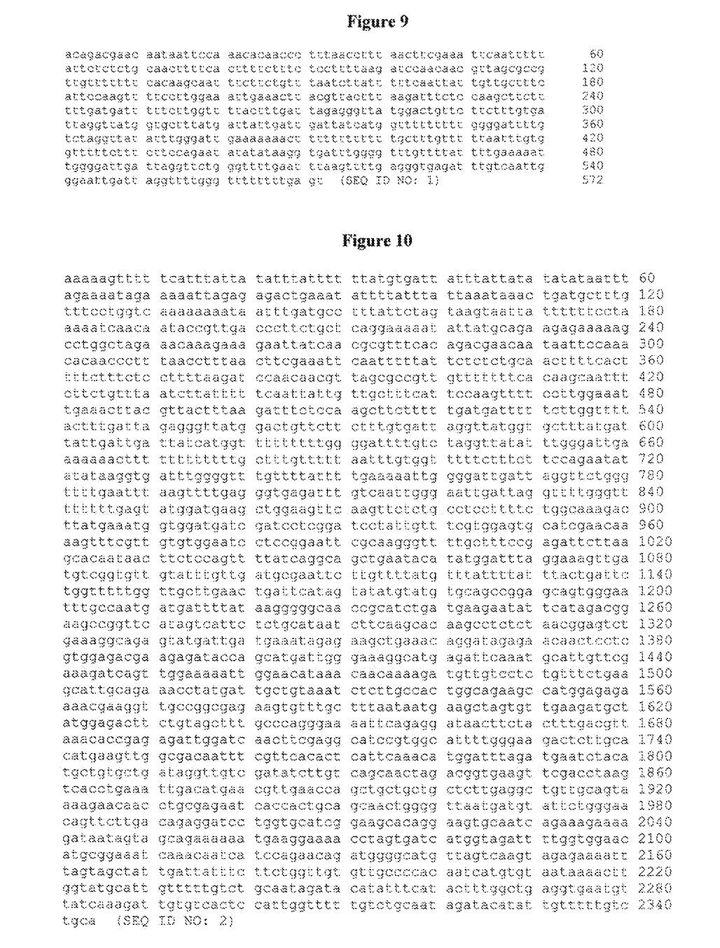

The term “5′-UTR” refers to the 5′-untranslated portion of the HsfA4 gene. For MsHsfA4, the 5′UTR is 572 nucleotides the sequence of which is illustrated in FIG. 9 (SEQ ID NO:1). The 5′-UTR is 741 nucleotides for PaHsfA4, the 5′-UTR is 284 nucleotides for NtHsfA4 and the 5′-UTR for AtHsf A4a is 511 nucleotides. As indicated in FIG. 2, the 5′-UTR sequence of the HsfA4 precedes the start codon (ATG).

In accordance with a method of enhancing the expression of a gene in a legume plant, a construct comprising at least a portion of the 5′-UTR of an HsfA4 gene, such as the 5′-UTR of the MsHSFA4 gene, is introduced into cells of the plant upstream of the gene to be expressed. The 5′-UTR portion of the HsfA4 functions as an enhancer to upregulate the expression of the gene to be expressed, e.g. heat shock gene, or a reporter gene such as a GUS reporter gene. As one of skill in the art will appreciate, one or more nucleotide modifications may be made to the 5′-UTR which do not affect the function of the 5′-UTR to enhance gene expression. In addition, the 5′-UTR may be truncated, e.g. from the 5′ end thereof, providing that the truncation does not abolish the function of the 5′-UTR of HsfA4 gene to upregulate gene expression. In one embodiment, the construct used in the present method retains at least the portion of the HsfA4 5′-UTR that includes an internal ribosome entry site (IRES), e.g. the gene sequence from position 461-573 of the MsHsfA4 5′-UTR (SEQ ID NO:4).

The HsfA4 5′-UTR gene construct, e.g. an MsHsfA4 gene construct, may be made using well-known techniques in the art of gene construct synthesis. In addition to the 5′-UTR or portion thereof, the construct may include additional nucleotide sequence, for example, an inert linker sequence, e.g. situated downstream of the 5′-UTR, and thus located between the 5′-UTR and the start codon of a gene to be expressed. In an alternative embodiment, the construct may itself include the gene to be expressed situated downstream of the 5′-UTR portion. Thus, the construct may include a gene, endogenous or exogenous to the plant into which the construct is introduced, for enhanced expression.

Moreover, once prepared, the construct is introduced into a plant for uptake by plant cells using recombinant technology that is well-established in the art. For example, the construct may be incorporated into a suitable vector which may then be introduced into cells of the plant, for example using the Agrobacterium method of plant transformation, for incorporation upstream of a gene to be expressed, i.e. upstream of the ATG start codon of the gene to be expressed. Transformed plant tissue is then selected for use to regenerate a plant therefrom which includes the HsfA4 construct.

The method provides a means to enhance the expression of any gene in a plant including, but not limited to endogenous or exogenous genes such as heat shock protein genes, such as genes encoding low molecular weight heat shock proteins, genes encoding enzymes associated with the Halliwell-Asada pathway (e.g. ascorbate peroxidase, glutathione reductase, superoxide dismutase, etc.) and reporter genes such as the GUS reporter gene. In one embodiment, enhanced gene expression in the presence of 5′-UTR of MsHsf4A was shown to correlate with the upregulated expression of the GUS reporter gene. It follows that any gene whose expression is enhanced by the gene sequence of the 5′-UTR of MsHsfA4 will demonstrate an upregulation in the translation of the corresponding mRNA of this gene.

In a further aspect of the invention, a recombinant legume plant cell is provided which is capable of enhancing the expression of a gene. The plant cell comprises a construct including at least a portion of the 5′-UTR of an HsfA4 gene, e.g. the 5′-UTR of the MsHsfA4 gene, upstream of the gene to be expressed. The plant cell, such as a plant cell from the Fabaceae family, e.g. Medicago sativa, Medicago trunculata or Lotus japonicus comprises a gene sequence which includes at least a portion of the 5′-UTR of an HsfA4 gene such as the MsHsfA4 gene. In one embodiment, the plant cell is a protoplast. In another embodiment, the plant cell comprises exogenous MsHsfA4-5′-UTR.

Embodiments of the invention are described in the following examples which are not to be construed as limiting.

### Example 1

### Materials and Methods

### Vector Construction

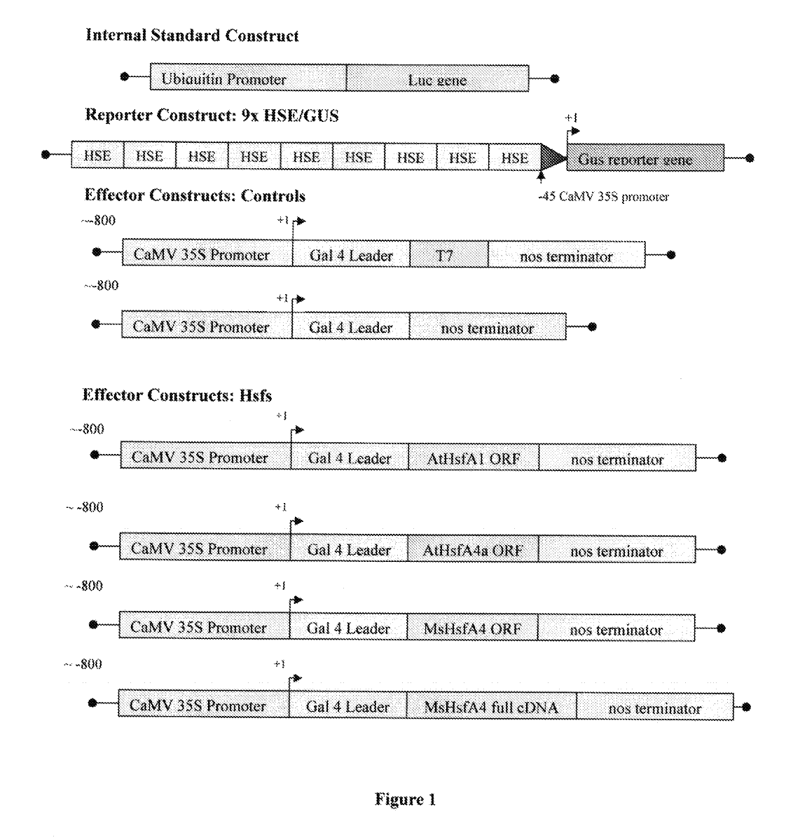

Expression constructs used in transfection experiments were kindly provided by Prof. Eva Czarnecka-Verner (University of Florida) and Dr. Jeremy Friedberg (Table 1 and FIG. 1). The constructs created were based on pBI121 (Clontech, Palo Alto, Calif.). Four effector vectors were used in the transient assay each containing differing coding regions of Hsfs; ORF of AtHsfA1 (also acting as a positive control), ORF of AtHsfA4, ORF of MsHsfA4 and complete cDNA of MsHsfA4 with 5′-UTR. Two control vectors; T7 epitope tag (amino acid sequence MASMTGGQQMG (SEQ ID No: 5) derived from pET17b) and Gal4 leader (derived from Saccharomyces cerevisiae) were used. Downstream from the promoter, the vector backbone contained a T7 tag or Gal 4 leader, Hsf coding region, and a poly-A-nos terminator sequence. Each construct was designed to activate β-Glucuronidase (GUS) reporter gene and was driven by a minimal 35S promoter (TATA box) fused to nine perfect (full consensus) synthetic core heat shock elements (HSE) (non-plant origin) (Table 1 and FIG. 1). GUS reporter activities were normalized using an internal luciferase standard which contained a luciferase coding sequence directed by a full length maize ubiquitin promoter (Ubi-Luc) (Table 1 and FIG. 1). The protoplast assays were repeated two to three times and were conducted in two to three replications.

TABLE 1

| | Internal | Reporter | | Origin/ |
|---|---|---|---|---|
| Construct | Standard | Vector | Effector Vector | Function |
| A | Ubi-Luc | 9 x HSE/GUS | T7 tag | Control |
| B | Ubi-Luc | 9 x HSE/GUS | Gal4 Leader | Control |
| C | Ubi-Luc | 9 x HSE/GUS | AtHSFA1 ORF | Arabidopsis |
| | | | | thaliana |
| D | Ubi-Luc | 9 x HSE/GUS | AtHSFA4a ORF | Arabidopsis |
| | | | | thaliana |
| E | Ubi-Luc | 9 x HSE/GUS | MsHSFA4 ORF | Medicago |
| | | | | sativa |
| F | Ubi-Luc | 9 x HSE/GUS | MsHSFA4 cDNA | Medicago |
| | | | (full length) | sativa |

### Vector Construction

The positive control, Gal4leader-AtHsfA1, was constructed from Gal4DBD-AtHsfA1 and Gal4leader-VP16 provided from Prof. Eva Czarnecka-Verner. The segment VP16 (acidic activation domain derived from herpes simplex virus) from the pGal4-VP16 was excised with the restriction enzymes SalI-NotI and then cloned into AtHsfA1 ORF that was obtained by polymerase chain reaction (PCR) from pGal4DBD construct using two synthetic primers with the SalI-NotI restriction sites. PCR products were produced under the following conditions; amplification occurred under the cycling conditions of denaturing at 94° C. for 1 minute, with an annealing temperature of 58° C., extension at 72° C. (using Taq DNA polymerase, Roche). PCR products were checked for quality using agarose gels and digested with SalI and NotI overnight at 37° C. Digested PCR products were purified with an ethanol precipitation by adding 2.5 volumes 100% ethanol and 1/10 volume 3M sodium acetate incubated at −20° C. for 2 hrs. Following incubation PCR products were centrifuged and resuspended in 20 μl of ddH₂O and quantified on an 0.8% agarose gel with ethidium bromide.

### Plasmid Preparation and Bacterial Transformation

Ligation of digested PCR products into digested cloning vector was accomplished using T4 DNA ligase (Invitrogen). Ligated vectors were electroporated into competent DH5a Escherichia coli cells and grown on plates that contained 100 μg/mL ampicillin, Luria broth (LB) media (1% w.v), Bactotryptone, 0.5% (w/v) yeast extract, and NaCl. Positive colonies were selected and grown to logarithmic phase of cell growth in standard Luria broth media supplemented with 100 μg/mL ampicillin at 37° C. with shaking at 255 rpm. Cells were pelleted by centrifugation at 1600×g and plasmid DNA was extracted using Qiagen Midi DNA™ extraction kit according to the manufacturer's instructions (Qiagen Inc.). Successful cloning was confirmed by agarose gel electophoresis, PCR and restriction digest to ensure the fragment was intact. Internal standard control and reporter plasmids (Luc and GUS, respectively) were grown in Escherichia coli cells and prepared as described above.

### Plant Tissue Growth

### Seed Sterilization and Germination

A wetting solution of 70% EtOH and 0.1% SDS (sodium dodecyl sulfate) surfactant was applied to Lotus japonicus and Arabidopsis seeds at room temperature for 5 minutes with agitation to enhance the sterilization process. Then a sterilizing solution of 20% bleach and 0.1% SDS was applied for another 8-10 minutes, then rinsed in sterile water. Germination occurred at 22° C., 16/8 hours day/night in a growth chamber (200-250 μE sec⁻¹ m⁻²) for 7 days on sterile wet filter paper in Petri dishes. For Medicago sativa and Medicago truncatula seeds, a 30% bleach sterilization solution with 0.1% Tween as a surfactant was applied for 5 minutes with agitation and then rinsed.

### Tobacco Protoplast Preparation

Sterile Nicotiana tabaccum L. (tobacco) plantlets were grown in tissue culture from leaf cuttings under aseptic conditions on Murashige and Skoog (MS) media, under a regime of 16 hours light/8 hours darkness and at a temperature of 20° C. Plantlets were grown to yield leaves which were approximately 3 to 5 cm in diameter. Each enzymatic digestion required four leaves to yield approximately one million protoplasts per mL. Under aseptic conditions four scored tobacco leaves were placed in a 15 mm Fisher culture plate covered with 30 mL of 0.5% Cellulysin and 0.2% Macerase enzyme (each obtained from Calbiochem) digestion solution in K3S media (Table 7). The upper and lower epidermis of each tobacco leaf was scored by tapping the surface using a scalpel. The leaf samples were incubated overnight at 28° C. in the absence of light on a shaker at 50 rpm.

### Medicago, Lotus, Arabidopsis Protoplast Preparation

Medicago sativa L. (alfalfa) plantlets along with Medicago truncatula, Lotus japonicus and Arabidopsis thaliana were grown from sterilized seed on Murashige and Skoog (MS) media, under a regime of 16 hours light/8 hours darkness and at a temperature of 20° C. Plantlets were grown to yield mature leaves. Each enzymatic digestion required 15 leaflets to yield approximately one million protoplasts per mL. Under aseptic conditions the upper epidermis of each leaflet was removed using forceps and a scalpel and placed in a 15 mm Fisher culture plate covered with 30 mL of a modified XU enzyme solution (Table 5). The leaf samples were incubated overnight at 28° C. in the absence of light on a shaker at 50 rpm.

### Protoplast Isolation

Digested leaf samples containing protoplast suspensions were filtered using sieves (45 μm) into sterile 15 mL glass centrifuge tubes. The tubes were covered and centrifuged in a fixed rotor at 40×g for 7 min. The pellet of protoplasts was removed with a glass Pasteur pipette and transferred to a new glass tube containing 9 mL of W5 wash solution and spun at 77×g for 10 min (Table 12). The pelleted protoplasts were then resuspended in 1 mL of K3M media (Table 8) and subsequent dilution was determined during viability determination and enumeration.

### Monitoring Protoplast Viability and Enumeration of Protoplasts

A volume of 100 μl Evan's blue (0.5% w/v in 0.7 M sorbitol) was added to 1 mL cell suspension of isolated protoplasts incubated for 10 min at room temperature, then centrifuged at 1000 rpm. The protoplasts were then resuspended in 1 mL of K3M media (Table 8) and observed on a glass slide under an Axiophat-Zeiss light microscope. Viable protoplasts excluded the Evan's blue dye and appeared clear or yellowish, while non viable cells appeared blue. The number of viable protoplast cells was determined with a hemocytometer in order to dilute the protoplast suspension to 1 million viable protoplasts/mL (as described in Friedberg et al, 2006, the relevant contents of which are incorporated herein by reference).

### Transient Transformation and Expression

Each transient transformation event was contained in a microfuge tube containing 3 plasmids including: luciferase internal standard (5 μg), effector construct (5 μg) and the reporter construct (5 μg). To each tube 50 μl of resuspended protoplasts (approximately 100 000) was aliquotted. An equal volume of 25% PEG solution (Table 11) was added, mixed gently and incubated for 30 min at room temperature. The transformation reaction was stopped by adding 900 μl of K3M solution and inverted to mix. The tubes were left to incubate at 28° C. overnight. Following incubation, microfuge tubes were spun at 15 000×g at 4° C. for 5 min and the supernatant was discarded. The pellet of cells was resuspended in 100 μl of GUS/Luc extraction buffer (Table 13) and ruptured by sonication (16 pulses at 50% power). Cellular debris was centrifuged at 15 000×g at 4° C. for 7 min and the supernatant was transferred to a new tube.

### Fluorometric β-Glucuronidase (GUS) Assays

Quantitative determination of GUS activity was accomplished by fluorometric GUS assay. The spectrofluorophotometer (Shimadzu, RF-5000, Kyoto, Japan) was calibrated with a fresh preparation of MU (7-hydroxy-4-methylcoumarin), as a calibration solution. GUS Reaction Buffer (Table 14) was incubated at 37° C. and then 50 μl of protein extract was added to 0.5 ml Reaction Buffer. At regular time intervals (0, 15, 30, and 60 min), successive 100 μl aliquots of sample were taken into Eppendorf tubes containing 0.9 ml Na₂CO₃ Stop Buffer to end the reaction. Fluorescence was subsequently determined using a spectrofluorophotometer with excitation at 365 nm and emission at 455 nm. The quantitative measurements of GUS activity was expressed as pmol methylumbelliferone (MU)/mg protein/min units. The fluorescence readings used in the below calculation were extrapolated from the linear portion of the curve at fixed time points and applied to the formula below to obtain the GUS activity. The normalized GUS activity was determined by dividing the GUS fluorescence value by the luciferase activity value for each sample, thus eliminating variation due to transformation efficiency.

### Normalized Value=GUS Value/Luc Value

### Luciferase Assay

In a black microtitre plate 5 μg of cell extract was aliquotted and placed into FLUOstar OPTIMA microplate reader (BMG Labtechnologies Inc.). Then 25 μl of chilled luciferin (Roche) was dispensed, mixed and the luciferase activity was normalized to total protein (Bradford Assay). Each sample was examined in duplicate in a single experiment and 6 independent experiments were performed.

### Bradford Assay

To ensure equivalent levels of protein were being used in the assays, each transformed protoplast supernatant was measured for protein concentration with Bovine Serum Albumin (BSA) as a standard according to Bradford (1976). The reaction was performed in a clear multi-well plate and read by a SpectraMax plus384 spectrophotometer (Molecular Devices, Sunnyvale, Calif.). Dilutions of the BSA (1 mg/ml) stock solution was aliquoted (0, 0.5, 1.0, 1.5, 2.0, 2.5, 3.0, 4.0 μg in 5 μl extraction buffer) (in triplicate). To this 5 μl of the Bradford solution was added to the multiwell plate for calibration. The absorbance was read at 600 nm and the protein concentration of the samples calculated using the BSA standard.

### Histochemical (X-Gluc) Test

A histochemical staining was performed as a check for GUS gene expression according to Jefferson (1987) with some modification. The substrate 5 bromo-4-3indolyl-B-D-glucuronide (X-gluc) was dissolved in DMSO (dimethyl sulfoxide, 20 mg mL-1) and added to the transformed protoplasts to give a final concentration of 0.5 mg mL-1 and incubated for 30 minutes. Visual inspection was conducted by Axiophat-Zeiss light microscope. Blue color formation occurred in the expression sites where X-gluc was catabolized by GUS gene product.

### Bioinformatics Analysis of 5′ Untranslated Region of MsHsfA4

The nucleotide blast program (Blastn) was used to search the 5′-UTR sequence of MsHSFA4 for similarities using Genbank from the National Center for Biotechnology Information (NCBI), http://www.ncbi.nlm.nih.gov/ with focus on plants (taxid:3193) and then a more focused search was conducted on the group Fabaceae (taxid:3803) to determine whether other long 5′UTR regions are present within the legume family. Other databases used to further search information included: 1) Plant transcription factor database (http://planttfdb.cbi.pku.edu.cn) and 2) German research centre for environmental health database (http://mips.helmholtz-muenchen.de/plant). A secondary program, UTRScan (http://www.baltb.cnr.it/BIG/UTRScan/) was also utilized to compare the 5′ untranslated region of MsHsfA4 to other 5′ untranslated regions in Hsf genes and determine potential regulatory motifs which may be present within the 5′-UTR of MsHsfA4.

### Statistical Analysis

All statistical analysis was performed using SAS (statistical analysis software, version 8.0, Cary California). All data were subjected to a variance analysis using the “proc glm” procedure of SAS (statistical analysis software). For this analysis the variance was partitioned into blocks and treatments. A natural log transformation of the data was required to obtain homogeneity and normal distribution of error variance. The type I error rate was set at 0.05.

### Results

### Optimization Protoplast Extraction

Obtaining protoplasts from alfalfa leaves was difficult. The enzymatic digestion protocol that was utilized for tobacco did not result in any digestion of the leaf. This problem was similarly experienced in plant species in Lotus and Medicago truncatula, even when younger plantlets were grown and utilized. This may have been due to the thicker cuticle present in these species of legumes. Therefore an improved and optimized digestion procedure was created to allow for ease in digestion. Firstly, leaves were selected with a 1 mm-3 mm stem. With two forceps one holding the edge of the stem, the lower epidermis was carefully removed. The peeled surface was placed on a modified enzymatic media (modified XU enzyme solution) that was also optimized for this protocol. Finally, with a very sharp scalpel the top epidermis was gently scored by slightly tapping the leaf to allow the enzyme to digest from the top as well.

### GUS/Luciferase Assay

The goal of these experiments was to determine the potential of the MsHsfA4 gene to activate transcription and determine the effect of the 5′UTR of MsHsfA4. Transient Hsf expression assays were conducted using tobacco (Nicotiana tabaccum), thale cress (Arabidopsis thaliana), alfalfa (Medicago sativa), Medicago truncatula, and Lotus japonicus mesophyll protoplasts. Six effector constructs were utilized in concert with 9×HSE/GUS reporter. Two effector control constructs, a Gal4 (untranslated) 5′ leader and T7 polypeptide tag, served as negative controls. Two AtHsf constructs, AtHSFA1a and AtHSFA4a were maintained as positive controls. Two MsHsfA4 constructs including: the open reading frame of MsHsf; and the full length cDNA of MsHSFA4 gene sequence, with a long (573 bp) 5′ untranslated region (5′-UTR) was utilized (FIG. 2). The internal standard construct was the ubiquitin (from maize) promoter driven luc gene. The endogenous protoplast HSF activity was accounted for by using pHSE9/GUS reporter alone (FIG. 1). Quantitative GUS activity was determined by measuring the fluorescence produced by conversion of MUG substrate to MU product by β-glucuronidase enzyme encoded by GUS gene, and the relative reporter gene activity was represented as the ratio of GUS to luciferase activity. A GUS histochemical preliminary analysis was conducted as a confirmation effort and a stronger staining intensity in those cells which had higher gene expression levels was observed (data not shown); this was supported by the fluorometric GUS data. A combined analysis of variance was conducted for relative GUS activity level. Analysis using SAS (statistical analysis software) revealed a significant species×treatment effect detected (P=0.0162), therefore a more detailed analysis utilizing a comparison of treatment effects (the Hsf constructs) within each species was performed (Table 3).

TABLE 3
Mean reporter gene expression (as measured by GUS: Luciferase activity ratio) and
statistical contrasts of treatments of four Hsf effector constructs and three negative controls
tested the 9x HSE/GUS reporter system in five different plant species protoplasts. Data
analyzed on a natural log scale and means back transformed to the original scale are provided.
Endo (Endogenous protoplasts), T7 (pT7 tag), G4 (Gal 4 leader), At1 (AtHsfA1) At4
(AtHsfA4), Ms4 (MsHsfA4a), U-Ms4 (MsHsfA4a + 5′UTR).

| | In Normalized | | | | | Back transformed Mean | | | | |
|---|---|---|---|---|---|---|---|---|---|---|
| | GUS/Luc | | | | | (Normalized GUS/Luc × 10⁻⁶) | | | | |
| | | | | Medicago | Lotus | | | | Medicago | Lotus |
| | Tobacco | Arabidopsis | Alfalfa | truncatula | japonicus | Tobacco | Arabidopsis | Alfalfa | truncatula | japonicus |
| Construct | | | | | | | | | | |
| Endo | 13.75 | 9.56 | 14.02 | 15.29 | 13.58 | 0.937 | 0.014 | 1.227 | 4.369 | 0.790 |
| T7 | 13.79 | 9.59 | 14.01 | 15.61 | 13.78 | 0.975 | 0.015 | 1.215 | 6.016 | 0.965 |
| G4 | 13.66 | 9.92 | 14.08 | 15.45 | 13.3 | 0.856 | 0.020 | 1.303 | 5.127 | 0.597 |
| At1 | 14.91 | 12.24 | 14.68 | 16.58 | 15.27 | 2.988 | 0.207 | 2.374 | 15.871 | 4.282 |
| At4 | 14.38 | 12.7 | 14.54 | 16.71 | 15.31 | 1.759 | 0.328 | 2.064 | 18.074 | 4.457 |
| Ms4 | 14.52 | 12.88 | 14.53 | 16.64 | 15.25 | 2.023 | 0.392 | 2.043 | 16.852 | 4.198 |
| U-Ms4 | 14.35 | 11.65 | 15.17 | 16.77 | 15.39 | 1.707 | 0.115 | 3.875 | 19.192 | 4.828 |
| se | 0.135 | 0.279 | 0.209 | 0.243 | 0.52 | | | | | |
| P-value | | | | | | | | | | |
| G4, T7 vs | 0.868 | 0.5780 | 0.9313 | 0.4221 | 0.9487 | | | | | |
| Endo | | | | | | | | | | |
| G4 vs T7 | 0.5001 | 0.4380 | 0.8027 | 0.6337 | 0.5288 | | | | | |
| At1, A4, | 0.0001 | 0.0001 | 0.0278 | 0.0001 | 0.0154 | | | | | |
| Ms4 vs | | | | | | | | | | |
| Endo | | | | | | | | | | |
| At1 vs | 0.0087 | 0.2773 | 0.6483 | 0.7107 | 0.9535 | | | | | |
| At4 | | | | | | | | | | |
| At4 vs | 0.4647 | 0.6604 | 0.9574 | 0.8364 | 0.9365 | | | | | |
| Ms4 | | | | | | | | | | |
| U-Ms4 vs | 0.3937 | 0.0143 | 0.0372 | 0.6937 | 0.8534 | | | | | |
| Ms4 | | | | | | | | | | |

Hsf Assayed in Nicotiana tabaccum (Tobacco) Protoplasts

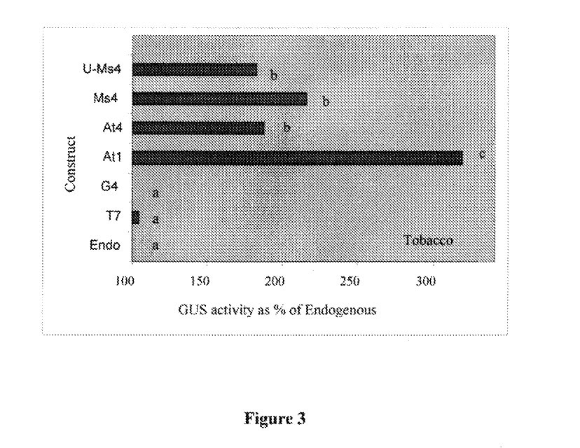

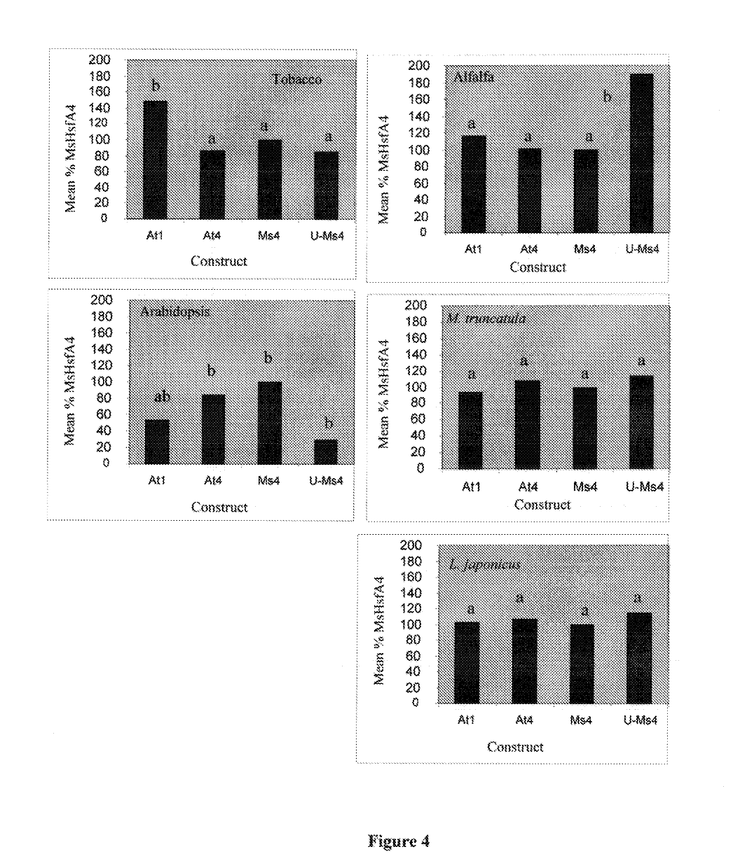

Various heat shock transcription factors were tested in five different protoplast systems to look for species specific effects. The two negative controls, Gal4 leader and the T7 tag constructs, each had negligible activity in Nicotiana tabaccum (tobacco) protoplasts indicating there was little to no background GUS activity present (FIG. 3). Low endogenous Hsf activity in tobacco protoplasts was also observed in this experiment; therefore the background tissue effects were negligible in tobacco (FIG. 3). The construct containing AtHsfA1 ORF from Arabidopsis class A1 Hsf served as a positive control for gene activation. The construct containing AtHsfA1 produced high levels of GUS activity in tobacco (FIG. 4 and Table 3 confirming its function as a bona fide Hsf. The relative reporter activity of the construct containing Arabidopsis HsfA4a (FIG. 4 and Table 3) compared to Arabidopsis HsfA1 was 51%. In the present study, the construct containing alfalfa MsHsfA4 ORF induced similar levels of GUS reporter activity as AtHsfA4a homologue (FIG. 4 and Table 3). In the case of the construct containing the 5′UTR of MsHsfA4, there was a numerical 16% decrease in reporter activity in comparison to the construct containing Medicago sativa HsfA4 ORF.

Hsf Assayed in Arabidopsis thaliana Protoplasts

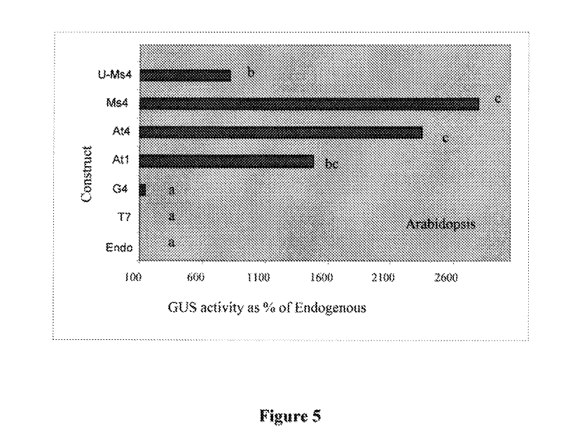

In Arabidopsis protoplasts the Gal4 leader and T7 tag control constructs had negligible activity indicating that there was little to no background GUS activity (FIG. 5). Low endogenous Hsf activity was observed in the Arabidopsis plant system (FIG. 5). The Arabidopsis class A1 Hsf construct served as a positive control for gene activation, and as anticipated, produced higher GUS reporter levels relative to the control constructs (FIG. 4 and Table 3). Therefore, the construct containing AtHsfA1 activated GUS gene expression. In the case of the Arabidopsis HsfA4a, the level of GUS gene expression were 45% higher compared to the levels for Arabidopsis HsfA1 (FIG. 4 and Table 3). The activity induced by the MsHsfA4 construct was 50% higher than the AtHsfA1 construct and numerically (17%) higher than the AtHsfA4 construct. However, as with the tobacco protoplast system, the inclusion of the 5′UTR in the MsHsfA4 construct resulted in a drastic inhibition of GUS reporter levels of approximately 70% (FIG. 4 and Table 3) in the Arabidopsis reporter cell system.

Hsf Assayed in Medicago sativa Protoplasts

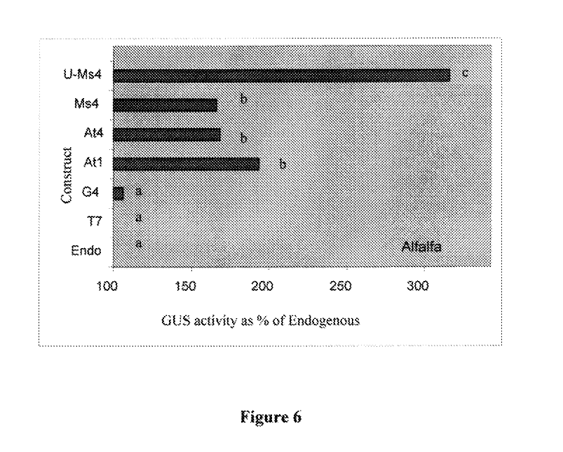

The results of the Hsf testing were quite different in alfalfa protoplasts. The two negative controls, Gal4 leader and the T7 tag constructs, showed negligible effect on GUS reporter activity. Low endogenous Hsf activity was also observed in this experiment (FIG. 6). However, all three Hsfs (AtHsfA1, AtHsfA4a and MsHsfA4) showed comparable GUS gene activity with AtHsfA1 being slightly above the other two Hsfs. Surprisingly, the construct containing MsHsfA4 showed a dramatically higher activity than the remaining constructs. Unlike in the tobacco and Arabidopsis systems, the presence of the 5′UTR in the construct containing MsHsfA4 did not reduce reporter gene expression; but on the contrary, it significantly increased the GUS reporter activity (FIG. 6 and Table 3). This indicates that the 5′UTR of MsHsfA4 has a regulatory effect on the expression of the MsHSFA4 construct that can be detected in the native alfalfa protoplasts as detected by the GUS reporter activity levels.

Hsf Assayed in Medicago truncatula Protoplasts

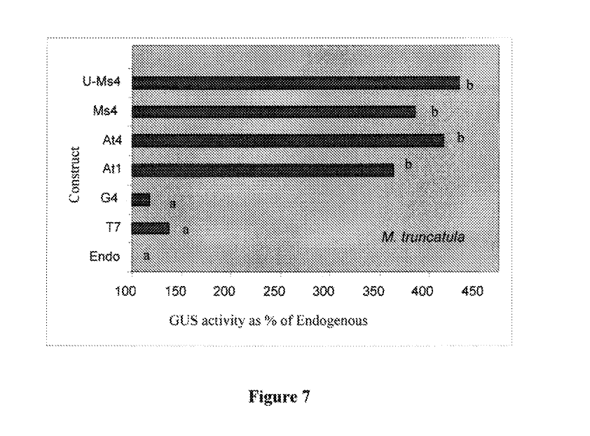

In Medicago truncatula protoplasts, the two negative controls, Gal4 leader and the T7 tag constructs had negligible activity (FIG. 7). The same as in tobacco, endogenous Hsf activity levels within the system indicated negligible effects (FIG. 7). The GUS reporter level with the construct containing AtHsfA1 that served as a positive control produced higher GUS levels observed relative to endogenous control and Gal 4 leader, T7 tag constructs (FIG. 7 and Table 3). Clearly, the construct containing AtHsfA1 is able to activate transcription in non-homologous protoplasts assays. Interestingly, all Hsf constructs displayed very similar transcription activity. Moreover, the MsHsfA4 construct containing the 5′UTR activated the GUS reporter gene the best as detected by the GUS reporter activity levels (FIG. 7 and Table 1).

Hsf Assayed in Lotus japonicus (Trefoil) Protoplasts

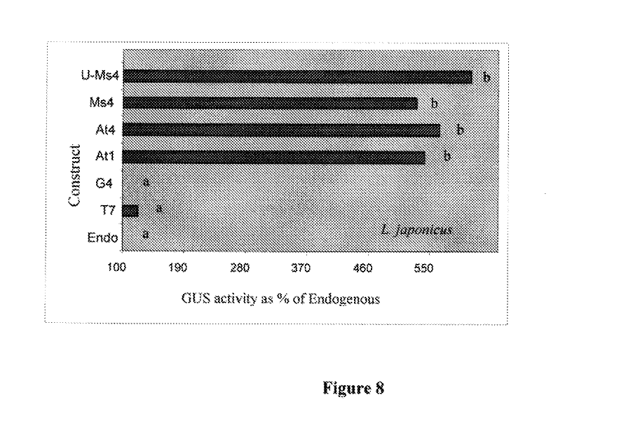

The Hsf activities when tested in Lotus protoplasts were almost identical to those in M. truncatula. The levels of activities as well as patterns of activities of individual Hsfs were mirrored. The response with the AtHsf4 construct was slightly higher than that of the AtHsfA1 construct and the MsHsfA4 construct, but the highest of all was GUS activity induced by the MsHsfA4 construct containing the 5′-UTR of MsHsfA4 (FIG. 4). All Hsf activities were basically the same, while control constructs Gal4 leader, T7 tag and endogenous Hsf activities were negligible (FIG. 8, Table 3).

### Bioinformatics Analysis of 5′ Untranslated Region

The use of bioinformatics has been instrumental in identifying putative transcription factor genes in plants. A BLAST search of the 5′-UTR gene sequence was conducted using Genbank (NCBI) to search for homology with references to the legume family. Strong homology with the 5′-UTR of MsHsfA4 (Medicago sativa) was found to that of a Medicago truncatula hypothetical protein (related Hsf gene), Lotus japonicus dynamin GTPase effector (Table 2). To a lesser degree, homologies were found with Medicago truncatula Heat shock protein homologous to DnaJ and Phaseolus acutifolius heat shock transcription factor mRNA (Table 2). Non coding 5′ untranslated regions have typically contained sequence motifs crucial for regulation and expression of genes (Pesole et al., 2000). Results from the UTRScan program indicated the presence of an internal ribosome entry site (IRES) within the 5′-UTR of MsHsfA4, located [461,572]: TTTGTT TTATTT TGAAA AA TTGGGGAT TGATTAG GTTCTGGG TTTTGAAT TTAAG TTTTGAGG GTGAG ATTTG TC AATTGG GAAT TGATT AGGTTTTG GGTTT TTTTTGAGT (Seq. ID No: 6). No other motifs or elements were detected.

TABLE 2
Genbank Sequences producing significant alignments with MsHSFA4 5′UTR

| | | Accession | Max |
|---|---|---|---|
| Organism | Details | No. | Score |
| Medicago sativa | Heat shock transcription factor mRNA | AF235958 | 1032 |
| | [HSFA4-6] | | |
| Medicago | BAC clone mth2-77l23 | AC152407 | 742 |
| truncatula | [hypothetical protein, related to Hsf gene] | | |
| Lotus japonicus | Genomic DNA, chromosome 4, clone: | AP004978 | 64.4 |
| | LjT44L05, TM0162a. | | |
| | [Dynamin GTPase effector] | | |
| Medicago | Clone mth2-20f2 | AC165135 | 46.4 |
| truncatula | [Heat shock protein DnaJ] | | |
| Phaseolus | Heat shock transcription factor mRNA | AY052627 | 46.4 |
| acutifolius | | | |
| Lotus japonicus | Genomic DNA, clone: LjT15P09, TM2024 | AP009790 | 44.6 |
| | [C2 calcium-dependent membrane targeting] | | |
| Glycine max | Clone BAC GM_WBb069O12 | EF533699 | 42.8 |
| | [lysin motif-type receptor-like kinases] | | |
| Medicago | Clone mth2-91j8, complete sequence | AC154089 | 42.8 |
| truncatula | [tandem CAA] | | |
| Lotus japonicus | Genomic DNA, chromosome 1, clone: | AP004537 | 42.8 |
| | LjT04C07, TM0098a | | |
| | [hypothetical protein, galactose-binding like; | | |
| | alpha-L-arabinofuranosidase] | | |
| Medicago | Chromosome 2 BAC clone mth2-77m24 | AC169181 | 41.0 |
| truncatula | [tandem TA] | | |
| Medicago | Clone mth2-170o9 | AC150786 | 41.0 |
| truncatula | [Zinc finger, RING-type] | | |

### Discussion

### Transient Protoplast Assay

The transient expression study of MsHsfA4 in mesophyll protoplasts utilized an endogenous Hsf background control and two types of constructs as negative controls: one with a transcript corresponding to the yeast Gal4 5′-leader and the other encoding the T7 epitope. The construct containing Gal4 leader sequence derived from yeast used in this study was to substitute for the native Hsf leader sequence in the Hsf ORF constructs. The control construct with the T7 tag, a peptide derived from bacteriophage T7 in this study served as a negative control expressing a small peptide only. These controls were used to maintain an equal number of promoter molecules in each protoplast transformation. These negative controls showed negligible transcriptional activities, indicating that the promoters and the construct backbones per se did not affect the GUS activity in protoplasts systems in tobacco (Nicotiana tabaccum), Arabidopsis thaliana, alfalfa (Medicago sativa), Medicago truncatula, and Lotus japonicus. This confirms the utility of the reporter system in plant mesophyll protoplasts. Thus comparisons of the various Hsf constructs would provide a reliable comparison of transcriptional activation potential.

In tobacco protoplasts, reporter activity with the Arabidopsis HsfA1 construct was found to be higher to that of the AtHsfA4 construct. However, this pattern is not reflected in other plant protoplast systems used in the present study. In the Arabidopsis protoplast system, Arabidopsis class A4 Hsf was found to be the higher of the two. This result which had Arabidopsis A4 higher expression than A1 was also detected in protoplast systems of M. truncatula and Lotus.

In addition, the constructs containing Arabidopsis class A4 Hsf and Medicago sativa MsHsfA4 ORF exhibited similar activity levels and profile patterns within M. sativa, M. truncatula and Lotus japonicus protoplast systems. Accordingly, legume protoplasts exhibit similar reporter levels in this regard.

In contrast, the construct containing MsHsfA4 ORF acted as a slightly stronger activator than the AtHsfA4 construct in the tobacco and Arabidopsis protoplast systems illustrating a pattern in which hsf activity depends on the type of species selected for transient reporter assays.

Previous studies based on expression levels with the MsHsfA4 ORF construct in comparison to the one containing the 5′-UTR of MsHsfA4 determined there was a reduction of activity in the presence of the latter in tobacco protoplast system. However, the present results illustrate that this is not the case in protoplast systems derived from legumes. The pattern in Arabidopsis (a reduced activity for MsHsfA4 containing the 5′-UTR) was found to be the same as in tobacco. However, in alfalfa the 5′-UTR did not reduce the MsHsfA4 construct activity. Quite opposite, it dramatically enhanced the expression of transactivator as shown by the upregulated GUS reporter signal. This enhancement was also detected in protoplasts from species of Lotus japonicus and M. truncatula.

### Bioinformatics Analysis of 5′ Untranslated Region

The 5′UTR gene sequence from MsHSFA4 was subjected to homology searches using the BLAST program from the National Center for Biotechnology Information (National Center for Biotechnology Information, Bethesda, Md. (Atschul et al. 1990) and also UTRscan from UTResource to determine further information pertaining to potential presence in UTR of functional elements by comparing nucleotide gene sequences to other user submitted sequence data for motifs defined in the UTRsite collection.

A BLAST search of the 5′UTR gene sequence from Medicago sativa was conducted using Genbank (NCBI) with a focus on homology found within the legume family. No other similar long UTRs were found, but regions of similarity were found within this family. Strong homology was found to that of a Medicago truncatula hypothetical protein (related to the Hsf gene) and Lotus japonicus derived dynamin GTPase effector. Mutation studies indicate that dynamin functions as a molecular regulator of receptor-mediated endocytosis (Sever et al. 1999). To a lesser degree, homologies were found with Medicago truncatula Heat shock protein DnaJ and Phaseolus acutifolius heat shock transcription factor mRNA.

The UTRScan delineated an element called an IRES (internal ribosome entry site) known to be involved in cap-independent expression of genes. Bioinformatic analysis revealed the presence of an IRES site within the 5′UTR of MsHsfA4. Typically this internal translation initiation mechanism has been observed in picornavirus, the 5′ UTR of plant infecting Tobomavirus, Antennapedia mRNA of Drosophila, Saccharomyceses cerevisiae mRNA and in human immunoglobulin heavy chain binding protein (BiP) mRNA. The 5′UTR of maize heat shock gene Hsp101 has also shown to exhibit IRES dependent activity. Cap-independent translation at the leader of Arabidopsis ribosomal protein S18 (RPS18C) also occurs. This mechanism is advantageous for the translation of mRNAs when cap-dependent initiation is impaired by events such as cell stress leading to apoptosis, or heat stress. This cap-independent expression of genes may be connected to the expression or enhancement of expression of the MsHsfA4 cDNA found in legumes such as Medicago. These segments such as the 5′ untranslated regions in genes may emerge as additional regulatory gene sequences for enhancing gene expression.

### CONCLUSION

The potential of a 5′ UTR of an HsfA4, such as MsHsfA4, to enhance gene expression was determined herein, and found to activate gene expression as represented by a GUS reporter system in protoplast systems of Medicago sativa (alfalfa), Medicago truncatula, Lotus japonicus (trefoil), Nicotiana tobaccum (tobacco) and Arabidopsis thaliana.

Distinct patterns were seen in the activity of the construct containing AtHsfA1 when compared to the construct containing AtHsfA4. In tobacco protoplast system, the reporter activity was significantly higher for the construct containing AtHsfA1, whereas in Arabidopsis, M. truncatula and Lotus the reverse was the case, with the construct containing AtHsfA4 being the higher of the two. The relative order of these patterns differed depending on the species, such that when the activity of the construct containing Arabidopsis Hsf class A4 activity was compared to the activity of the construct containing Medicago Hsf class A4, GUS reporter activation profiles showed similar activity levels in M. sativa, M. truncatula and Lotus protoplasts. There were negative effects of the 5′-UTR in tobacco and Arabidopsis plant systems (not legumes), and significantly positive effects in alfalfa, and numerical increases in the other legumes studied.

TABLE 4
Cellulysin/macerase protoplast enzyme digesting solution

| Component | Supplier/Catalogue Number | Concentration | For 100 ml | |
|---|---|---|---|---|
| Enzyme | | | | |
| Cellulase | Calbiochem-Cellulysin | 0.5% | 0.5 | g |
| | Cat# 219466 | | | |
| Macerase | Calbiochem-Macerase | 0.2% | 0.2 | g |
| | Cat# 441201 | | | |
| Media | | | | |
| K3S | | | 100 | ml |

Prepare fresh and sterile filter (store excess in −20° C.)

TABLE 5
Modified XU enzyme protoplast digesting solution

| Chemical/Enzyme | Amount | |
|---|---|---|
| Mannitol | 13 | g |
| CPW x 2 | 50 | ml |
| (Table 6) | | |
| Rhozyme | 2 | g |
| Meicellase | 4 | g |
| Macerozyme | 30 | mg |

pH 5.6
Bring volume to 100 ml then spin @3500 for 5 minutes, then add 50 ml K3S Media

TABLE 6
CPW (cell protoplast washing) Media

| Chemical | Amount |
|---|---|
| KH₂PO₄ | 13.6 mg |
| KNO₃ | 50.5 mg |
| CaCl2•2H₂O | 740.0 mg |
| MgSO₄•7H₂O | 120.0 mg |
| KI | 0.08 mg |
| | (100 ul of 83 mg/100 ml stock) |

pH 5.6, Autoclave

TABLE 7
K3S Media

| | | | | | | | | Working |
|---|---|---|---|---|---|---|---|---|
| Chemical | For 1 L | | For 2 L | | [Stock] | Stock prep | [Working] | 1x |
| Sugar | | | | | | | | |
| Components | | | | | | | | |
| sucrose | 136.9 | g | 272.8 | g | | | 0.4M | |
| Salts | | | | | | | | |
| Components | | | | | | | | |
| KNO₃ | 10 | ml | 20 | ml | 100x | 62.5 g/250 ml | 24.73 μM | 2.5 g/L |
| MgSO₄•7H₂O | 10 | ml | 20 | ml | 100x | 6.25 g/250 ml | 1.01 μM | 0.25 g/L |
| NaH₂PO₄•H₂O | 10 | ml | 20 | ml | 100x | 3.75 g/250 ml | 1.09 μM | 0.15 g/L |
| (NH₄)₂SO₄ | 10 | ml | 20 | ml | 100x | 3.35 g/250 ml | 1.01 μM | 0.134 g/L |
| CaCl₂•2H₂O | 10 | ml | 20 | ml | 100x | 22.5 g/250 ml | 6.12 μM | 0.9 g/L |
| m-Inositol | 10 | ml | 20 | ml | 100x | 2.5 g/250 ml | 0.56 μM | 0.1 g/L |
| K3 Supplement | 10 | ml | 20 | ml | | | | |
| (microelements) | | | | | | | | |
| (Table 10) | | | | | | | | |
| NH₄NO₃ | 10 | ml | 20 | ml | 100x | 6.0 g/250 ml | 3.0 μM | 0.24 g/L |
| FeSO₄•EDTA | 5 | ml | 10 | ml | 200x | | | |
| (Table 9) | | | | | | | | |
| Buffer | | | | | | | | |
| Components | | | | | | | | |
| MES | 10 | ml | 20 | ml | 0.5M | | 5.0 μM | |
| | | | | | pH5.7-5.8 | | | |
| Hormones | | | | | | | | |
| NAA | 1 | ml | 2 | ml | 1 mg/ml | | | |
| BA | 200 | μl | 400 | μl | 1 mg/ml | | | |
| Vitamins | | | | | | | | |
| B1 | 1 | ml | 2 | ml | 10 mg/ml | | | |
| B6 | 100 | μl | 200 | μl | 10 mg/ml | | | |
| NSA | 100 | μl | 200 | μl | 10 mg/ml | | | |

Notes:
NAA (α-naphthaleneacetic acid)
B1 (thiamine hydrochloride)
B6 (pyridoxol hydrochloride)
NSA (nicotinic acid C₆H₆N₂O MW122.13)
Additional Notes:
Adjust final pH to 5.7-5.8 via diluted KOH. Filter sterilize solutions and store in 40 ml aliquots (store excess in −20° C.)

TABLE 8
K3M Media

| | | | | | | | | Working |
|---|---|---|---|---|---|---|---|---|
| Chemical | For 1 L | | For 2 L | | [Stock] | Stock prep | [Working] | 1x |
| Sugar | | | | | | | | |
| sucrose | 30 | g | 60 | g | | | | |
| mannitol | 73 | g | 146 | g | | | | |
| Salts | | | | | | | | |
| KNO₃ | 10 | ml | 20 | ml | 100x | 62.5 g/250 ml | 24.73 μM | 2.5 g/L |
| MgSO₄•7H₂O | 10 | ml | 20 | ml | 100x | 6.25 g/250 ml | 1.01 μM | 0.25 g/L |
| NaH₂PO₄•H₂O | 10 | ml | 20 | ml | 100x | 3.75 g/250 ml | 1.09 μM | 0.15 g/L |
| (NH₄)₂SO₄ | 10 | ml | 20 | ml | 100x | 3.35 g/250 ml | 1.01 μM | 0.134 g/L |
| CaCl₂•2H₂O | 10 | ml | 20 | ml | 100x | 22.5 g/250 ml | 6.12 μM | 0.9 g/L |
| m-Inositol | 10 | ml | 20 | ml | 100x | 2.5 g/250 ml | 0.56 μM | 0.1 g/L |
| K3 Supplement | 10 | ml | 20 | ml | | | | |
| (microelements) | | | | | | | | |
| (Table 10) | | | | | | | | |
| NH₄NO₃ | 10 | ml | 20 | ml | 100x | 6.0 g/250 ml | 3.0 μM | 0.24 g/L |
| FeSO₄•EDTA | 5 | ml | 10 | ml | 200x | | | |
| (Table 9) | | | | | | | | |
| Buffer | | | | | | | | |
| MES | 10 | ml | 20 | ml | 0.5M | | 5.0 μM | |
| | | | | | pH5.7-5.8 | | | |
| Hormones | | | | | | | | |
| NAA | 1 | ml | 2 | ml | 1 mg/ml | | | |
| BA | 200 | μl | 400 | μl | 1 mg/ml | | | |
| Vitamins | | | | | | | | |
| B1 | 1 | ml | 2 | ml | 10 mg/ml | | | |
| B6 | 100 | μl | 200 | μl | 10 mg/ml | | | |
| NSA | 100 | μl | 200 | μl | 10 mg/ml | | | |

Notes:
NAA (α-naphthaleneacetic acid)
B1 (thiamine hydrochloride)
B6 (pyridoxol hydrochloride)
NSA (nicotinic acid C₆H₆N₂O MW122.13)
Adjust final pH to 5.7-5.8 via diluted KOH Filter sterilize and store in 2 ml aliquots (store excess in −20° C.)

TABLE 9
FeSO₄•EDTA Solution

| | | | 1x | |
|---|---|---|---|---|
| Chemical | For 250 ml | 200x Stock | working | [working] |
| FeSO₄•7H₂O | | 1.39 g/250 ml | 27.8 mg/L | 0.1 μM |
| Na₂EDTA•2H₂O | | 1.86 g/250 ml | 3.2 mg/L | 0.1 μM |

Filter sterilize

TABLE 10
K3 Supplement (Microelements)

| Chemical | For 500 ml | 100x Stock | 1x Working | [Working] |
|---|---|---|---|---|
| KI | | 37.5 mg/500 ml | 0.75 mg/L | 4.52 μM |
| MnSO₄•H₂O | | 500 mg/500 ml | 10 mg/L | 59.16 μM |
| ZnSO₄•7H₂O | | 100 mg/500 ml | 2 mg/L | 6.96 μM |
| H₃BO₃ | | 150 mg/500 ml | 3 mg/L | 48.52 μM |
| Na₂MoO₄•2H₂O | | 12.5 mg/500 ml | 0.25 mg/L | 1.03 μM |
| CoCl₂•6H₂O | | 1.25 mg/500 ml | 0.025 mg/L | 0.11 μM |
| CuSO₄ | | 1.25 mg/500 ml | 0.025 mg/L | 0.15 μM |

Filter sterilize (store excess at 4° C.)

TABLE 11
25% PEG for Transfection

| Chemical | For 20 ml | [Working] |
|---|---|---|
| PEG 6000 | 5.0 g | 25% |
| mannitol | 1.64 g | 0.45M |
| Ca(NO₃)₂•4H₂O | 0.47 g | 0.1M |

pH to 6.0
Filter sterilize and store in 2 ml aliquots (store excess in −20° C.)

TABLE 12
W5 Washing Solution

| Chemical | 1 Litre | |
|---|---|---|
| CaCl₂•6H₂O | 27.4 | g |
| Or | | |
| CaCl₂•2H₂O | 18.4 | g |
| NaCl | 9.0 | g |
| KCl | 0.4 | g |
| Glucose | 1.0 | g |
| 0.5M MES pH | 10 | ml |
| 5.7-5.8 | | |

Filter sterilize and store in 40 ml aliquots (store excess in −20° C.)

TABLE 13
GUS/LUC Extraction Buffer

| | For 100 ml | | | |
|---|---|---|---|---|
| Chemical | (from stock) | | [Stock] | [Working] |
| Na₂HPO₄ pH 7.0 | 5 | ml | 1M | 50 mM |
| EDTA (Na₂) pH 8.0 | 2 | ml | 0.5M | 10 mM |
| N-laurylsacrosine (Na salt) | 286 | μl | 35% | 0.1% |
| Triton X-100 | 1 | ml | 10% | 0.1% |

Add 1 μl/ml of β-mercaptoethanol (10 mM) just before use (store excess in −20° C.)

TABLE B14
GUS Reaction Buffer

| Chemical | Concentration |
|---|---|
| GUS/LUC Extraction Buffer | |
| MUG | 22 mg/25 ml |
| 4-Methylumbelliferyl β-D-glucuronide | |
| sigma cat#: M-5664 | |

Add 1 μl/ml of β-mercaptoethanol (10 mM) just before use
Note:
MUG is light sensitive - wrap in foil.

We claim:

1. A method of enhancing the expression of a gene to be expressed in a legume plant comprising introducing a construct comprising at least a portion of the 5′-UTR of MsHsfA4 into the cells of said plant upstream of the gene to be expressed.

2. The method of claim 1, wherein the gene is a reporter gene.

3. The method of claim 2, wherein the reporter gene is a GUS reporter gene.

4. The method of claim 1, wherein the legume plant is a plant of the Fabaceae family.

5. The method of claim 4, wherein the legume plant is selected from the group consisting of Medicago ssp., Medicago sativa, Medicago truncatula, Phaseolus acutifolius, Phaseolus ssp., Lotus japonicus, Trifolium ssp., Pisum sativum and Glycine max.

6. The method of claim 5, wherein the legume plant is Medicago sativa.

7. The method of claim 5, wherein the legume plant is Medicago truncatula.

8. The method of claim 5, wherein the legume plant is Lotus japonicus.

9. The method of claim 1, wherein the 5′-UTR comprises a sequence that encodes an internal ribosome entry site.

10. The method of claim 1, wherein the 5′-UTR is truncated to nucleotide 461.

11. The method of claim 1, wherein the 5′-UTR comprises the gene sequence of SEQ ID No: 1.

12. The method of claim 1, wherein the 5′-UTR has the sequence of SEQ ID No: 1.

13. A recombinant legume plant cell capable of exhibiting enhanced expression of a gene, wherein said cell comprises a construct comprising at least a portion of the 5′-UTR of MsHsfA4 gene upstream of the gene to be expressed.

14. The plant cell of claim 13, wherein the gene is a reporter gene.

15. The plant cell of claim 14, wherein the reporter gene is a GUS reporter gene.

16. The plant cell of claim 13, selected from a legume plant from the group consisting of Medicago ssp., Medicago sativa, Medicago truncatula, Phaseolus acutifolius, Phaseolus ssp., Lotus japonicus, Trifolium ssp., Pisum sativum and Glycine max.

17. The plant cell of claim 13, wherein the 5′-UTR comprises a sequence that encodes an internal ribosome entry site.

18. The plant cell of claim 13, wherein the 5′-UTR is truncated to nucleotide 461.

19. The plant cell of claim 13, wherein the 5′-UTR comprises the gene sequence of SEQ ID No: 1.

20. The method of claim 1, wherein the construct comprises the gene to be expressed downstream of the 5′-UTR portion.
